(19) United States
(12) Patent Application Publication
(10) Pub. No.: US 2022/0160534 A1
(43) Pub. Date: May 26, 2022

(54) CONSCIOUSNESS DISTURBANCE REDUCING APPARATUS AND CONSCIOUSNESS DISTURBANCE REDUCING METHOD

(71) Applicant: NIPPON TELEGRAPH AND TELEPHONE CORPORATION, Tokyo (JP)

(72) Inventors: Shingo TSUKADA, Musashino-shi, Tokyo (JP); Hiroshi NAKASHIMA, Musashino-shi, Tokyo (JP); Toichiro GOTO, Musashino-shi, Tokyo (JP)

(73) Assignee: NIPPON TELEGRAPH AND TELEPHONE CORPORATION, Tokyo (JP)

(21) Appl. No.: 17/440,881

(22) PCT Filed: Mar. 13, 2020
(86) PCT No.: PCT/JP2020/010992

§ 371 Date: Sep. 20, 2021

(30) Foreign Application Priority Data
Mar. 22, 2019 (JP) 2019-055453

(51) Int. Cl. A61F 5/34; A61M 16/10; A61M 16/12

(52) U.S. Cl. CPC A61F 5/34; A61M 2205/3303; A61M 16/12; A61M 16/1005

(57) ABSTRACT

An aspect of the present invention is a consciousness disorder mitigation apparatus, including: a first estimation unit configured to estimate body fluid volume information, the body fluid volume information being information on a body fluid volume present in a head of a user; a second estimation unit configured to estimate oxygen supply volume information, the oxygen supply volume information being information on an oxygen supply volume representing an amount of oxygen in a brain of the user; a pressurization unit configured to be attached to the user and to apply a pressure corresponding to an estimation result of the first estimation unit and an estimation result of the second estimation unit; and an oxygen supply unit configured to supply oxygen to the user based on the estimation result of the second estimation unit.

### TECHNICAL FIELD

The present invention relates to a consciousness disorder mitigation apparatus and a consciousness disorder mitigation method.

### BACKGROUND ART

Along with transition to an ultra-aging society, patients suffering from conditions such as cerebrovascular disorder, heart disease and arteriosclerosis have recently been increasing, mainly in people in their late seventies and older. Such patients suffering from conditions like cerebrovascular disorder, heart diseases and arteriosclerosis are considered to have high possibility of occurrence of a consciousness disorder caused by cerebral ischemia or a consciousness disorder due to change in acceleration, such as hemodynamic brain infarction and asymptomatic cerebral infarction (Non-Patent Literatures 1 and 2).

### CITATION LIST

### Non-Patent Literature

Non-Patent Literature 1: Toshihiko Iwamoto, Kiuchi Akihiro, “Cerebrovascular diseases in the elderly on a geriatric viewpoint”, Proceeding of the 45th Japanese Geriatrics Society Conference, <Plenary Lecture>, Journal of Japanese Geriatrics, 2003; 40: 476-479
Non-Patent Literature 2: Fujishima Masatoshi, “Heart diseases as risk factor of cerebrovascular diseases”, Japanese Circulation Society Journal, Circulatory Specialist, Vol. 6, No. 1, pp. 19-26

### SUMMARY OF THE INVENTION

### Technical Problem

A consciousness disorder is likely to lead to a severe accident if it occurs during an operation such as driving. Accordingly, it is desirable to have a technique that suppresses the occurrence of a consciousness disorder of a patient suffering from conditions such as cerebrovascular disorder, heart diseases, and arteriosclerosis.

In addition, the issue of occurrence of a consciousness disorder is not only related to patients suffering from cerebrovascular disorder, heart diseases, arteriosclerosis or the like but is also a common issue to patients with blood pressure adjustment dysfunction, such as Shy-Drager syndrome and diabetic neuropathy. Since a patient with blood pressure adjustment dysfunction such as Shy-Drager syndrome and diabetic neuropathy can have a fainting fit in response to a relatively minor change in acceleration such as standing up motion or ascent of an elevator, a technique to suppress a consciousness disorder is desirable. Further, the issue of occurrence of a consciousness disorder is also a common issue to operators who are subjected to a strong change in acceleration, such as racing car drivers and airplane pilots.

In view of these circumstances, the present invention is aimed at providing a technique for suppressing the occurrence of a consciousness disorder.

### Means for Solving the Problem

An aspect of the present invention is a consciousness disorder mitigation apparatus including: a first estimation unit configured to estimate body fluid volume information, the body fluid volume information being information on a body fluid volume present in a head of a user; a second estimation unit configured to estimate oxygen supply volume information, the oxygen supply volume information being information on an oxygen supply volume representing an amount of oxygen in a brain of the user; a pressurization unit configured to be attached to the user and to apply a pressure corresponding to an estimation result of the first estimation unit and an estimation result of the second estimation unit; and an oxygen supply unit configured to supply oxygen to the user based on the estimation result of the second estimation unit.

An aspect of the present invention is the consciousness disorder mitigation set forth above, further including: a partial pressure control unit configured to adjust a partial pressure of oxygen gas and a partial pressure of non-oxygen gas in mixture gas based on the oxygen supply volume information as the estimation result of the second estimation unit, the mixture gas being mixed gas of the oxygen gas which is oxygen in gas form and the non-oxygen gas which is gas of a different composition from the oxygen. The oxygen supply unit supplies the mixture gas adjusted by control of the partial pressure control unit to the user.

An aspect of the present invention is the consciousness disorder mitigation set forth above, in which the second estimation unit estimates the oxygen supply volume information based on oxygen concentration in expiration or inspiration of the user.

An aspect of the present invention is the consciousness disorder mitigation set forth above, in which the second estimation unit estimates the oxygen supply volume as the oxygen supply volume information, and when a state in which the oxygen supply volume estimated by the second estimation unit is lower than a predetermined volume continues for a predetermined period of time or longer, the oxygen supply unit supplies oxygen in gas form at a predetermined pressure to the user.

An aspect of the present invention is a consciousness disorder mitigation method including: a first estimation step of estimating body fluid volume information, the body fluid volume information being information on a body fluid volume present in a head of a user; a second estimation step of estimating oxygen supply volume information, the oxygen supply volume information being information on an oxygen supply volume representing an amount of oxygen in a brain of the user; a pressurization step of applying a pressure corresponding to an estimation result from the first estimation step and an estimation result from the second estimation step to the user; and an oxygen supply step of supplying oxygen to the user based on the estimation result from the second estimation step.

### Effects of the Invention

The present invention can provide a technique for suppressing the occurrence of a consciousness disorder.

### BRIEF DESCRIPTION OF DRAWINGS

FIG. 1 shows a usage example of a consciousness disorder mitigation apparatus 1 according to a first embodiment.

FIG. 2 shows an example of a pressurization unit 15 in the first embodiment.

FIG. 3 shows an example of a functional configuration of the consciousness disorder mitigation apparatus 1 in the first embodiment.

FIG. 4 is a flowchart showing a flow of specific processing executed by the consciousness disorder mitigation apparatus 1 in the first embodiment.

FIG. 5 is a flowchart showing a flow of specific processing of pressurization control processing in the first embodiment.

FIG. 6 shows an example of flow of specific processing executed by the consciousness disorder mitigation apparatus 1 with a first safety mechanism in a variant.

FIG. 7 shows an example of functional configuration of the consciousness disorder mitigation apparatus 1 with a second safety mechanism in a variant.

FIG. 8 is a flowchart showing a flow of specific processing where the consciousness disorder mitigation apparatus 1 in the first embodiment includes the second safety mechanism and pressurizes the neck of a user.

FIG. 9 shows an example of functional configuration of a consciousness disorder mitigation apparatus 1a in a second embodiment.

FIG. 10 is a flowchart showing an example of flow of processing executed by the consciousness disorder mitigation apparatus 1a in the second embodiment.

FIG. 11 shows an example of functional configuration of the consciousness disorder mitigation apparatus 1 with a pressurization control unit 143a in a variant.

### DESCRIPTION OF EMBODIMENTS

### First Embodiment

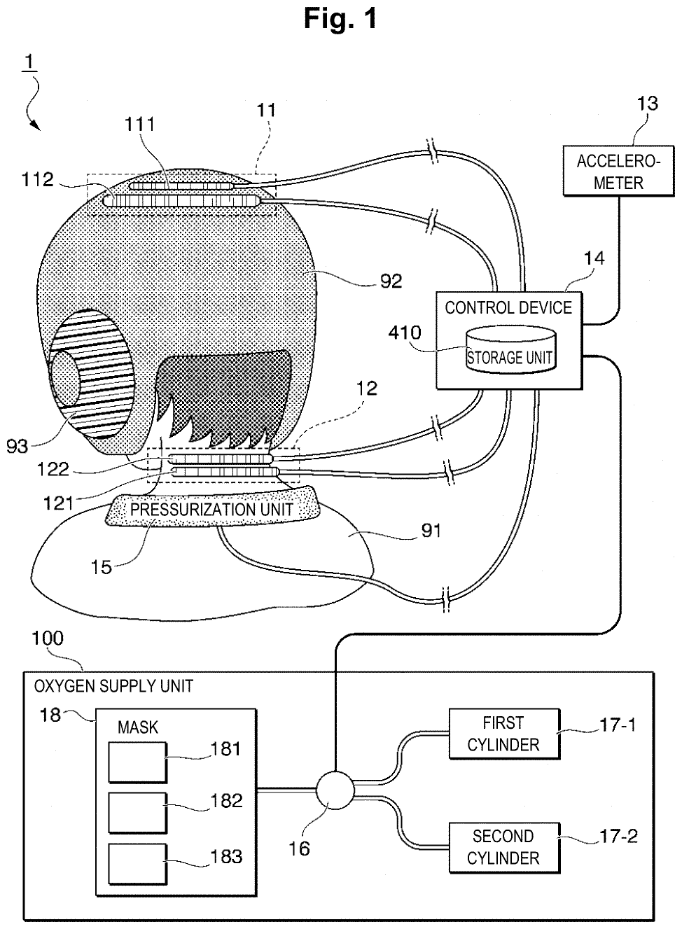

FIG. 1 shows a usage example of a consciousness disorder mitigation apparatus 1 according to a first embodiment. The consciousness disorder mitigation apparatus 1 in the first embodiment estimates body fluid volume and its variation amount in a skull by applying a current or a voltage to a user 91's head via electrodes and acquiring an impedance of the head. The consciousness disorder mitigation apparatus 1 in the first embodiment applies a pressure based on the estimated body fluid volume and its variation amount to the user 91's neck to suppress descending of blood, and maintains the body fluid volume in the head of the user 91 at an appropriate volume. Blood volume is an amount having high correlation with the body fluid volume in the skull. Accordingly, the body fluid volume in the skull may be the volume of blood in the skull, for example.

The consciousness disorder mitigation apparatus 1 in the first embodiment includes a first electrode portion 11, a second electrode portion 12, an accelerometer 13, a control device 14, a pressurization unit 15, and an oxygen supply unit 100.

The first electrode portion 11 has electrodes that make contact with the head of the user 91. More particularly, the first electrode portion 11 includes a first application electrode 111 and a first measurement electrode 112. The first measurement electrode 112 lies between the first application electrode 111 and the second electrode portion 12. The first application electrode 111 and the first measurement electrode 112 may be in contact with the head of the user 91 in any manner as long as they are in contact with the head. For example, the first application electrode 111 and the first measurement electrode 112 may make contact with the head of the user 91 wearing a helmet 92 by being attached to a top portion of the helmet 92, for example. Alternatively, the first application electrode 111 and the first measurement electrode 112 may make contact with temples of the user 91 by being attached to the helmet 92 and ear muffs 93 of a headphone. The helmet 92 and the ear muffs 93 of the headphone cover the temples of the user 91, so that the first application electrode 111 and the first measurement electrode 112 come into contact with the temples of the user 91. The first application electrode 111 and the first measurement electrode 112 may also make contact with the head of the user 91 wearing a face mask by being attached to a top portion of the face mask, for example. The helmet 92 is an impact-absorbing protector used in an environment which is subjected to an impact. The helmet 92 not only exists for the contact of the first application electrode 111 and the first measurement electrode 112 with the user 91 but also serves as a head protection member for protecting the user 91 present in an impact-subjected environment.

Alternatively, the user 91 may bring the first application electrode 111 and the first measurement electrode 112 into contact with himself by putting on a hood-shaped cloth bearing the first application electrode 111 and the first measurement electrode 112 thereon and wearing the helmet 92 over the cloth.

Between the first application electrode 111 and the second electrode portion 12, a voltage is applied for measuring the impedance of the head of the user 91. Thus, in a case where the first measurement electrode 112 is present between the first application electrode 111 and the second electrode portion 12, the impedance of the head is measured with high accuracy compared to where the first measurement electrode 112 is not present between the first application electrode 111 and the second electrode portion 12.

Note that the first measurement electrode 112 being present between the first application electrode 111 and the second electrode portion 12 means the following. Specifically, being present between the first application electrode 111 and the second electrode portion 12 means that a portion of the first measurement electrode 112 is present on a line H between a center point of the first application electrode 111 and a center point of the second electrode portion 12, where the line H is a curve connecting between the center point of the first application electrode 111 and the center point of the second electrode portion 12 and being parallel to a surface of the user 91's head.

However, the first measurement electrode 112 need not necessarily be present between the first application electrode 111 and the second electrode portion 12; it may be present at any location at which the impedance of the head can be measured, with consideration of impedance measurement accuracy.

For the consciousness disorder mitigation apparatus 1 in the first embodiment, the first measurement electrode 112 is preferably present between the first application electrode 111 and the second electrode portion 12.

The second electrode portion 12 includes electrodes to make contact with the neck of the user 91. More particularly, the second electrode portion 12 includes a second application electrode 121 and a second measurement electrode 122. The second measurement electrode 122 may be present between the second application electrode 121 and the first electrode portion 11. The second electrode portion 12 need not necessarily make contact with the neck but may make contact with any location at which the impedance of the head can be measured. For example, the second electrode portion 12 may be attached to an abdomen, waist, or buttocks of the user 91.

Between the second application electrode 121 and the first electrode portion 11, a voltage is applied for measuring the impedance of the head of the user 91. Thus, in a case where the second measurement electrode 122 is present between the second application electrode 121 and the first electrode portion 11, the impedance of the head is measured with high accuracy compared to where the second measurement electrode 122 is not present between the second application electrode 121 and the first electrode portion 11.

Note that the second measurement electrode 122 being present between the second application electrode 121 and the first electrode portion 11 means the following. Specifically, being present between the second application electrode 121 and the first electrode portion 11 means that a portion of the second measurement electrode 122 is present on a line I between a center point of the second application electrode 121 and a center point of the first electrode portion 11, where the line I is a curve connecting between the center point of the second application electrode 121 and the center point of the first electrode portion 11 and being parallel to the surface of the user 91's head.

However, the second measurement electrode 122 need not necessarily be present between the second application electrode 121 and the first electrode portion 11; it may be present at any location at which the impedance of the head can be measured, with consideration of impedance measurement accuracy.

For the consciousness disorder mitigation apparatus 1 in the first embodiment, the second measurement electrode 122 is preferably present between the second application electrode 121 and the first electrode portion 11.

The accelerometer 13 measures an acceleration acting on itself and outputs a signal indicating a magnitude of the acceleration (hereinafter referred to as an “acceleration signal”). The accelerometer 13 may be present at any location as long as it is set at a location subjected to an acceleration approximately the same as the acceleration acting on the user 91.

For example, the accelerometer 13 may be attached to the helmet 92 worn by the user 91, or may be attached to an object subjected to an acceleration approximately the same as that acting on the user 91, such as a seat in an airplane on which the user 91 is aboard.

The control device 14 includes a processor, memories, a storage unit 410 and the like connected by a bus, and executes a program. The control device 14 functions as a device via execution of the program. The storage unit 410 is composed using a storage device such as a magnetic hard disk device or a semiconductor storage device. The storage unit 410 stores various information related to the consciousness disorder mitigation apparatus 1. The control device 14 is electrically connected with the first electrode portion 11 and the second electrode portion 12 and controls the first electrode portion 11 and the second electrode portion 12. The control device 14 performs control such that the pressurization unit 15 applies a pressure of a predefined level to a location in contact with the pressurization unit 15 on the basis of currents flowing in the first electrode portion 11 and the second electrode portion 12 and an acceleration signal outputted by the accelerometer 13.

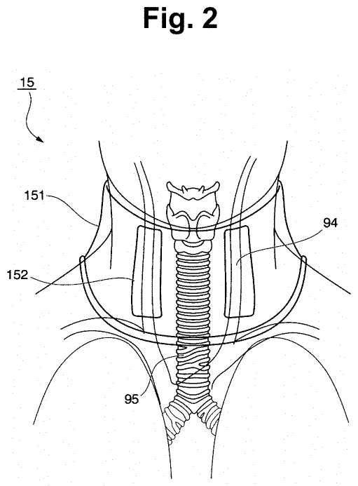

The pressurization unit 15 makes contact with the user 91's body and applies a pressure to the user 91 under control of the control device 14. The pressurization unit 15 may be set at any location on the body as long as it pressurizes the body of the user 91. For example, if the pressurization unit 15 is set in the neck of the user, decrease in the body fluid volume in the skull of the user 91 is suppressed by the application of a pressure to the neck of the user 91, which suppresses an occurrence of an anomalous state in the user 91 such as low vision, unconsciousness and central nervous disorder. The pressurization unit 15 may be any object that applies a pressure to the user 91. For example, the pressurization unit 15 may be an air bag or an elastic band. Also, in a case where the pressurization unit 15 is set in the neck, the pressurization unit 15 is preferably one that selectively pressurizes an internal jugular vein. For selective pressurization of the internal jugular vein in the neck, the pressurization unit 15 may be of the shape shown in FIG. 2, for example.

FIG. 2 shows an example of the pressurization unit 15 in the first embodiment.

The pressurization unit 15 shown in FIG. 2 includes a neck pressurization unit 151 and two front neck bladders 152. The neck pressurization unit 151 is a neck collar or a neck pillow. In FIG. 2, the neck pressurization unit 151 is in a deployed state. The deployed state is a state in which the neck pressurization unit 151 is inflated. The front neck bladders 152 are air bags vertically long in the vicinity of sternomastoid muscle. The front neck bladders 152 are located in two vertical rows near the sternomastoid muscle. The front neck bladders 152 each pressurize an internal jugular vein 94. The pressurization unit 15 is preferably placed so as to avoid a central portion where a trachea 95 runs in terms of avoiding compression of the airway. Thus, the front neck bladders 152 are preferably located at a position where they do not compress the trachea 95. With such pressurization, outflow of blood from the head of the user 91 to his trunk can be delayed and loss of consciousness can be suppressed.

Returning to FIG. 1, its description is continued.

The oxygen supply unit 100 supplies gaseous oxygen (hereinafter referred to as “oxygen gas”) to the user 91 under control of the control device 14. The oxygen supply unit 100 includes a control valve 16, a first cylinder 17-1, a second cylinder 17-2, and a mask 18. The oxygen supply unit 100 is controlled for its operation by the control device 14. Specifically, operation of the control valve 16 is controlled by the control device 14. The control valve 16 is connected by a tube which feeds gas to the first cylinder 17-1, the second cylinder 17-2 and the mask 18. The first cylinder 17-1 is a source that supplies oxygen in gas form (hereinafter referred to as “oxygen gas”) to the mask 18 via the control valve 16. The second cylinder 17-2 is a source that supplies gas which is gas other than oxygen gas and which is not harmful to the user 91 (hereinafter referred to as “non-oxygen gas”) to the mask 18 via the control valve 16. The non-oxygen gas may be any gas that is of a different composition from the oxygen gas and does no harm to the user 91. The non-oxygen gas is nitrogen or nitrogen with carbon dioxide added, for example.

The control valve 16 mixes the oxygen gas supplied from the first cylinder 17-1 to the mask 18 and the non-oxygen gas supplied from the second cylinder 17-2 to the mask 18 and supplies it to the mask 18 under control of the control device 14. In the following description, gas made by mixture of the oxygen gas and the non-oxygen gas will be called mixture gas. The control valve 16 controls a concentration of the oxygen gas and a concentration of the non-oxygen gas in the mixture gas under control of the control device 14. The control valve 16 controls a pressure of the mixture gas under control of the control device 14.

The mask 18 includes a gas emission unit 181 and a gas sensor 182. The gas emission unit 181 is connected by a tube that feeds mixture gas to the control valve 16 and emits the mixture gas supplied via the control valve 16. The gas sensor 182 measures oxygen concentration in expiration or inspiration of the user 91. Non-oxygen concentration in expiration or inspiration refers to the concentration of the component other than oxygen in expiration or inspiration. In the following description, a result of measurement by the gas sensor 182 will be called a ventilatory status measurement result.

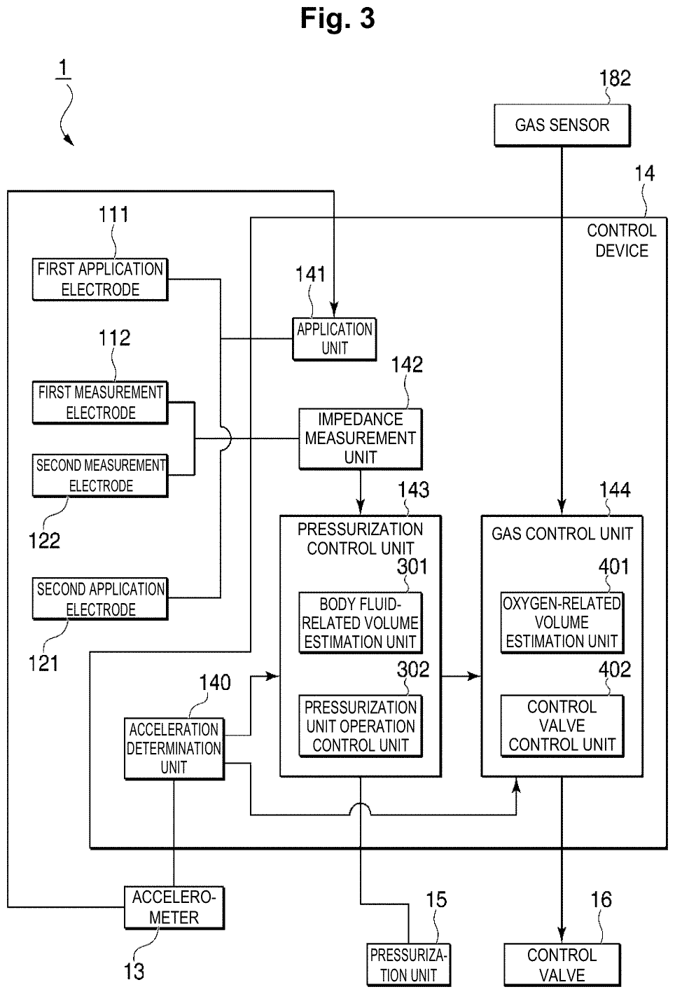

FIG. 3 shows an example of functional configuration of the consciousness disorder mitigation apparatus 1 in the first embodiment.

The control device 14 includes an acceleration determination unit 140, an application unit 141, an impedance measurement unit 142, a pressurization control unit 143 and a gas control unit 144.

The acceleration determination unit 140 determines whether the acceleration indicated by the acceleration signal outputted by the accelerometer 13 is less than a predefined value (hereinafter referred to as a “reference acceleration”) or not.

The application unit 141 applies a voltage between the electrodes of the first application electrode 111 and the second application electrode 121. The application unit 141 may be anything that can apply a voltage between the electrodes of the first application electrode 111 and the second application electrode 121. For example, the application unit 141 voltage may be a voltage source.

The impedance measurement unit 142 measures the impedance between the first measurement electrode 112 and the second measurement electrode 122 by acquiring currents flowing in the first measurement electrode 112 and the second measurement electrode 122.

The pressurization control unit 143 controls the pressure applied by the pressurization unit 15 to the user 91 based on the determination result of the acceleration determination unit 140 and the impedance measured by the impedance measurement unit 142.

The pressurization control unit 143 includes a body fluid-related volume estimation unit 301 and a pressurization unit operation control unit 302. The body fluid-related volume estimation unit 301 executes body fluid-related volume estimation processing. By executing the body fluid-related volume estimation processing, the body fluid-related volume estimation unit 301 estimates body fluid volume information, which is information on the body fluid volume in the skull of the user 91, based on the impedance measured by the impedance measurement unit 142. A variation amount of the body fluid volume is venous return volume, for example. The body fluid volume information may include the body fluid volume in the skull of the user 91. The body fluid volume information may include the variation amount of the body fluid volume per unit time.

For simplicity of explanation, the following description assumes that the body fluid volume information includes the body fluid volume in the skull of the user 91 and the variation amount of the body fluid volume per unit time. In the following description, the body fluid volume in the skull of the user 91 estimated by the body fluid-related volume estimation unit 301 will be referred to as an estimated body fluid volume. In the following description, the variation amount of body fluid volume in the skull of the user 91 per unit time estimated by the body fluid-related volume estimation unit 301 will be referred to as an estimated body fluid variation amount.

The pressurization unit operation control unit 302 controls the operation of the pressurization unit 15 based on the body fluid volume and the variation amount of the body fluid volume per unit time estimated by the body fluid-related volume estimation unit 301 when the determination result of the acceleration determination unit 140 is a determination result that the acceleration indicated by the acceleration signal is equal to or higher than a reference acceleration. Specifically, the pressurization unit operation control unit 302 first determines a pressurization unit operation. The pressurization unit operation is an operation which the pressurization control unit 143 makes the pressurization unit 15 perform and which is related to the pressure applied to the user 91. The pressurization unit operation is an operation of increasing, decreasing, or maintaining the pressure applied to the user 91, for example. The pressurization unit operation control unit 302 then makes the pressurization unit 15 perform the pressurization unit operation determined.

The pressurization unit operation control unit 302 makes the pressurization unit 15 perform a reference pressurizing operation when the determination result of the acceleration determination unit 140 is a determination result that the acceleration indicated by the acceleration signal is less than the reference acceleration. The reference pressurizing operation is an operation of the pressurization unit 15 applying a predefined reference pressure to the user 91. That is, the reference pressurizing operation is an operation of the pressurization unit 15 that changes the amount of pressurization applied by the pressurization unit 15 to the user 91 to a predefined reference value. The reference pressure may be 0 pa, for example.

When the body fluid volume in the skull is low, less current flows into the skull and hence the impedance of the head becomes large compared to when the body fluid volume is high. Accordingly, when the impedance measured by the impedance measurement unit 142 is equal to or greater than a predefined impedance, for example, the estimated body fluid volume estimated by the body fluid-related volume estimation unit 301 will be low.

In the following description, the processing that is executed by the body fluid-related volume estimation unit 301 and the pressurization unit operation control unit 302 when the acceleration indicated by the acceleration signal outputted by the accelerometer 13 is equal to or greater than the reference acceleration will be referred to as pressurization control processing.

The gas control unit 144 controls a partial pressure of the oxygen gas and a partial pressure of the non-oxygen gas in the mixture gas emitted from the gas emission unit 181 based on the determination result of the acceleration determination unit 140 and a ventilatory status measurement result, which is a measurement result of the gas sensor 182.

The gas control unit 144 includes an oxygen-related volume estimation unit 401 and a control valve control unit 402. The oxygen-related volume estimation unit 401 executes oxygen-related volume estimation processing. By executing the oxygen-related volume estimation processing, the oxygen-related volume estimation unit 401 estimates oxygen supply volume information, which is information on an amount of oxygen in the user 91's brain (hereinafter referred to as “oxygen supply volume”), based on the ventilatory status measurement result. The oxygen supply volume information may include the oxygen supply volume. The oxygen supply volume information may include a variation amount of the oxygen supply volume per unit time.

For the simplicity of explanation, the following description assumes that the oxygen supply volume information includes the oxygen supply volume and the variation amount of the oxygen supply volume per unit time. In the following description, the oxygen supply volume estimated by the oxygen-related volume estimation unit 401 will be referred to as an estimated oxygen supply volume. In the following description, the variation amount of the oxygen supply volume per unit time estimated by the oxygen-related volume estimation unit 401 will be referred to as an estimated oxygen variation amount.

The control valve control unit 402 controls the partial pressure of the oxygen gas and the partial pressure of the non-oxygen gas in the mixture gas emitted from the gas emission unit 181 based on the estimated oxygen supply volume and the estimated oxygen variation amount, which are estimation results estimated by the oxygen-related volume estimation unit 401. Specifically, the control valve control unit 402 controls the partial pressure of the oxygen gas and the partial pressure of the non-oxygen gas in the mixture gas emitted from the gas emission unit 181 by controlling the operation of the control valve 16 based on the estimated oxygen supply volume and the estimated oxygen variation amount estimated by the oxygen-related volume estimation unit 401. Control by the control valve control unit 402 for increasing the partial pressure of the oxygen gas is bolus injection.

The control valve control unit 402 executes reference partial pressure control when the determination result of the acceleration determination unit 140 is a determination result that the acceleration indicated by the acceleration signal is less than the reference acceleration. The reference partial pressure control is control executed by the control valve control unit 402. Here, the control valve control unit 402 controls the control valve 16 to set the partial pressure of the oxygen gas in the mixture gas to a first reference partial pressure and set the partial pressure of the non-oxygen gas to a second reference partial pressure. The first reference partial pressure is a magnitude of a physical quantity in the same dimension as the partial pressure of the oxygen gas and is a predefined magnitude of a physical quantity. The second reference partial pressure is a magnitude of a physical quantity in the same dimension as the partial pressure of the non-oxygen gas and is a predefined magnitude of a physical quantity. That is, the reference partial pressure control is control that adjusts the partial pressure of the oxygen gas and the partial pressure of the non-oxygen gas to predefined reference values.

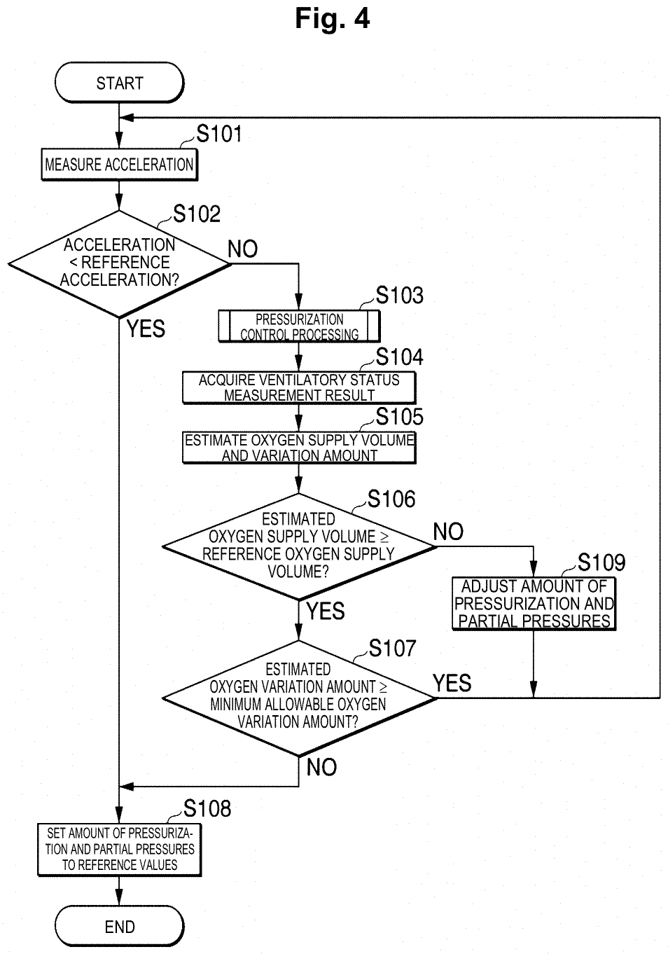

FIG. 4 is a flowchart showing a flow of specific processing executed by the consciousness disorder mitigation apparatus 1 in the first embodiment. In the following description, the processing shown in FIG. 4 will be referred to as first consciousness disorder mitigation processing.

The accelerometer 13 measures the acceleration and outputs an acceleration signal (step S101). The acceleration determination unit 140 acquires the acceleration signal and determines whether the acceleration indicated by the acceleration signal is less than the reference acceleration or not (step S102).

If the acceleration is equal to or greater than the reference acceleration (step S102: No), the pressurization control unit 143 executes the pressurization control processing (step S103).

After step S103, the oxygen-related volume estimation unit 401 acquires a ventilatory status measurement result, which is the measurement result of the gas sensor 182 (step S104). After step S104, the oxygen-related volume estimation unit 401 acquires the estimated oxygen supply volume and the estimated oxygen variation amount based on the ventilatory status measurement result (step S105). Acquiring the estimated oxygen supply volume and the estimated oxygen variation amount based on the ventilatory status measurement result means estimating the oxygen supply volume and the variation amount of the oxygen supply volume per unit time.

The control valve control unit 402 determines whether the estimated oxygen supply volume obtained in step S105 is equal to or greater than a reference oxygen supply volume or not (step S106). The reference oxygen supply volume is a magnitude of a physical quantity in the same dimension as the estimated oxygen supply volume and is a predefined magnitude of a physical quantity.

If the estimated oxygen supply volume is equal to or greater than the reference oxygen supply volume (step S106: YES), the control valve control unit 402 then determines whether the estimated oxygen variation amount is equal to or greater than a reference oxygen variation amount or not (step S107). The reference oxygen variation amount is a magnitude of a physical quantity in the same dimension as the estimated oxygen variation amount and is a predefined magnitude of a physical quantity.

If the estimated oxygen variation amount is equal to or greater than the reference oxygen variation amount (step S107: YES), the flow returns to the processing in step S101. If the estimated oxygen variation amount is less than the reference oxygen variation amount (step S107: NO), the pressurization unit operation control unit 302 makes the pressurization unit 15 perform the reference pressurizing operation and the control valve control unit 402 executes the reference partial pressure control (step S108).

If the estimated oxygen supply volume is less than the reference oxygen supply volume in step S106 (step S106: NO), the pressurization unit operation control unit 302 makes the pressurization unit 15 perform a pressurization adjusting operation and the control valve control unit 402 executes partial pressure adjustment control (step S109). The pressurization adjusting operation is an operation of the pressurization unit 15 that sets the pressure applied to the user 91 to a pressure appropriate for the estimated oxygen supply volume and the estimated oxygen variation amount. The partial pressure adjustment control is control that sets the partial pressure of the oxygen gas and the partial pressure of the non-oxygen gas respectively to pressures appropriate for the estimated oxygen supply volume and the estimated oxygen variation amount by controlling the control valve 16.

The partial pressures of the oxygen gas and the partial pressure of the non-oxygen gas appropriate for the estimated oxygen supply volume and the estimated oxygen variation amount refer to such partial pressures that increase the concentration of oxygen in the mixture gas to a predetermined value or higher when the estimated oxygen supply volume is lower than the predetermined value, for example. More specifically, they refer to the partial pressure of the oxygen gas and the partial pressure of the non-oxygen gas such that a ratio between the partial pressure of the oxygen gas and the partial pressure of the non-oxygen gas in the mixture gas is a predetermined value or higher.

After step S109, the flow returns to the processing in step S101.

If the acceleration is less than the reference acceleration in step S102 (step S102: YES), the processing in step S108 is performed. In step S108, if the pressurization unit 15 is not applying a pressure to the user 91, the pressure of the pressurization unit 15 will not lower any more.

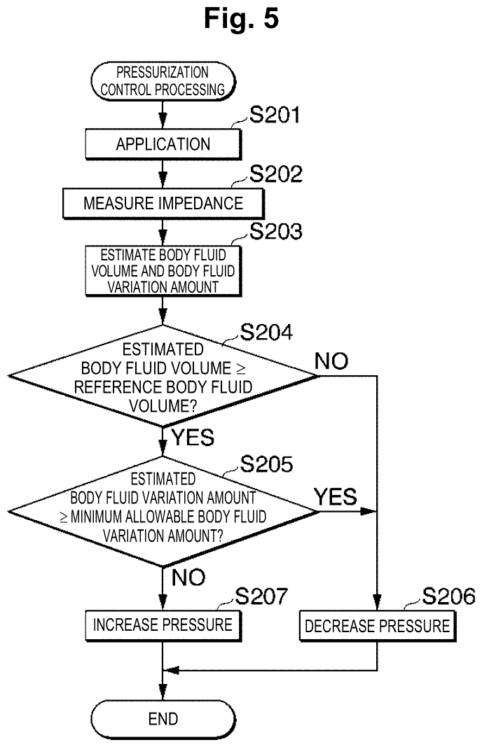

FIG. 5 is a flowchart showing a flow of specific processing of the pressurization control processing in the first embodiment.

The application unit 141 applies a voltage between the electrodes of the first application electrode 111 and the second application electrode 121 (step S201). The impedance measurement unit 142 acquires currents flowing in the first measurement electrode 112 and the second measurement electrode 122. The currents flowing in the first measurement electrode 112 and the second measurement electrode 122 are currents generated by the voltage applied by the application unit 141. The impedance measurement unit 142 measures the impedance between the first measurement electrode 112 and the second measurement electrode 122 based on a current value of the acquired current and the voltage applied by the application unit 141 (step S202).

The pressurization control unit 143 acquires the impedance measured by the impedance measurement unit 142 and estimates the body fluid volume in the skull of the user 91 and its variation amount per unit time (step S203). The pressurization control unit 143 determines whether the estimated body fluid volume is equal to or greater than a predefined body fluid volume (hereinafter referred to as a “reference body fluid volume”) (step S204). If the estimated body fluid volume is equal to or greater than the reference body fluid volume (step S204: YES), the pressurization control unit 143 determines whether the estimated body fluid variation amount is equal to or greater than a predefined value (hereinafter referred to as a “minimum allowable body fluid variation amount”) (step S205). The minimum allowable body fluid variation amount is an amount indicating that the user 91 has high risk of having a disorder such as low vision, loss of consciousness and central nervous disorder if the estimated body fluid variation amount is equal to or greater than that amount. If the estimated body fluid variation amount is equal to or greater than the minimum allowable body fluid variation amount (step S205: YES), the pressurization control unit 143 controls the pressurization unit 15 to apply a pressure to the user 91 (step S206).

If the estimated body fluid volume is less than the reference body fluid volume in step S204 (step S204: NO), the pressurization control unit 143 controls the pressurization unit 15 to apply a pressure to the user 91 (step S206).

If the estimated body fluid variation amount is less than the minimum allowable body fluid variation amount in step S205 (step S205: NO), the pressurization control unit 143 controls the pressurization unit 15 to lower the pressure applied by the pressurization unit 15 to the user 91 (step S109). If the pressurization unit 15 is not applying a pressure to the user 91, the pressure of the pressurization unit 15 will not lower any more.

The consciousness disorder mitigation apparatus 1 of the first embodiment configured as described above includes the pressurization control unit 143 and the gas control unit 144. Thus, the consciousness disorder mitigation apparatus 1 can suppress the occurrence of a consciousness disorder due to low-oxygen brain state, such as loss of consciousness caused by a large acceleration.

Note that the mixture gas need not necessarily contain only two kinds of gases with different compositions. The mixture gas may contain three or more kinds of gases with different compositions.

In order to reduce the possibility of the user 91 falling into any of the dangerous conditions mentioned above, it is desirable that the consciousness disorder mitigation apparatus 1 is equipped with a safety mechanism (hereinafter referred to as a “first safety mechanism”), which promptly decreases the amount of pressurization when excessive accumulation of body fluid is detected from an impedance value. Excessive accumulation of body fluid means body fluid being accumulated in the skull beyond an allowable body fluid volume. The allowable body fluid volume is a minimum body fluid volume which is accumulated in the skull and which places a human body in a dangerous condition. The human body is in a dangerous condition when the amount of body fluid accumulated in the skull is higher than the allowable body fluid volume. The first safety mechanism may function such that the pressurization control unit 143 determines whether the estimated body fluid volume is equal to or greater than the allowable body fluid volume and if the estimated body fluid volume is equal to or greater than the allowable body fluid volume, the pressurization control unit 143 decreases the amount of pressurization, for example.

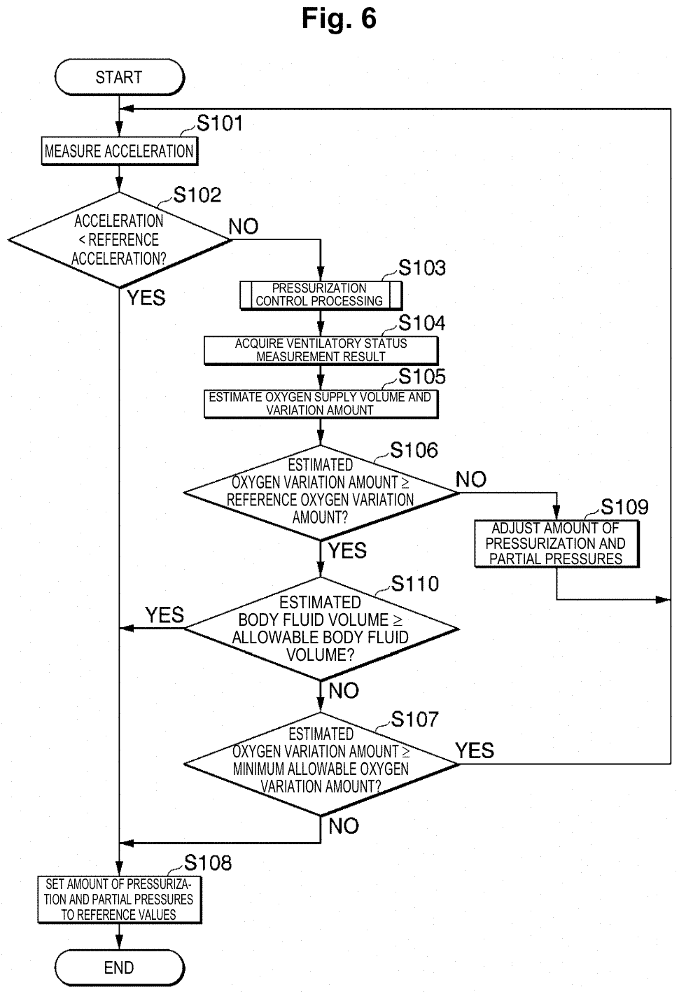

FIG. 6 shows an example of flow of specific processing executed by the consciousness disorder mitigation apparatus 1 with the first safety mechanism in a variant. The processing shown in FIG. 6 will be referred to as second consciousness disorder mitigation processing below.

The flowchart shown in FIG. 6 is different from the flowchart shown in FIG. 4 in that step S110 is executed between step S106 and step S107.

In the following description, for simplicity of explanation, description of similar processing to those executed by the consciousness disorder mitigation apparatus 1 in the first embodiment is omitted by giving the same reference numerals as in FIG. 4.

After step S106, the pressurization control unit 143 determines whether the estimated body fluid volume is equal to or greater than the allowable body fluid volume or not (step S110).

If the estimated body fluid volume is equal to or greater than the allowable body fluid volume in step S110 (step S110: YES), the processing in step S108 is executed. If the estimated body fluid volume is less than the allowable body fluid volume in step S110 (step S110: NO), the processing in step S107 is executed.

The consciousness disorder mitigation apparatus 1 with the first safety mechanism in the variant configured as described above can reduce the possibility of the user 91 falling into any of the dangerous conditions mentioned above by including the first safety mechanism. FIG. 6 has been thus far described.

If a pressure is continuously applied to the neck from the pressurization unit 15 for a long time, excessive body fluid can accumulate in the head or the neck and the user 91 can be placed in a dangerous condition. Thus, it is desirable that the consciousness disorder mitigation apparatus 1 further include a timer 150 in addition to the functional components of FIG. 3 and include a safety mechanism (hereinafter referred to as a “second safety mechanism”) that automatically decreases the amount of pressurization by the pressurization unit 15 after a certain period of time (hereinafter referred to as “limit time”) has passed since the start of pressurization as measured by the timer.

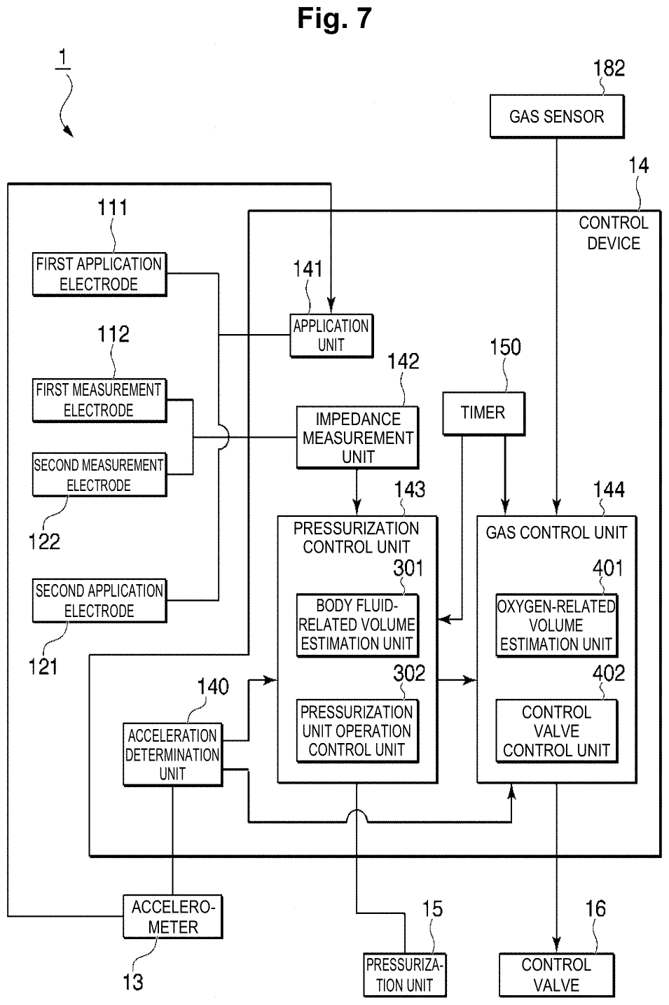

FIG. 7 shows an example of functional configuration of the consciousness disorder mitigation apparatus 1 with the second safety mechanism in a variant. In the following, description of components with similar functions to those of the consciousness disorder mitigation apparatus 1 in the first embodiment is omitted by giving the same reference numerals.

The consciousness disorder mitigation apparatus 1 with the second safety mechanism includes the timer 150 in addition to the functional components of FIG. 2. The timer 150 measures time from a time origin, where the time origin is the time at which the consciousness disorder mitigation apparatus 1 starts pressurization to the user 91. The timer 150 outputs the measured time to the pressurization control unit 143 and the gas control unit 144. The timer 150 measures time from a time origin, where the time origin is the time at which the pressurization control unit 143 starts controlling the pressurization unit 15, for example.

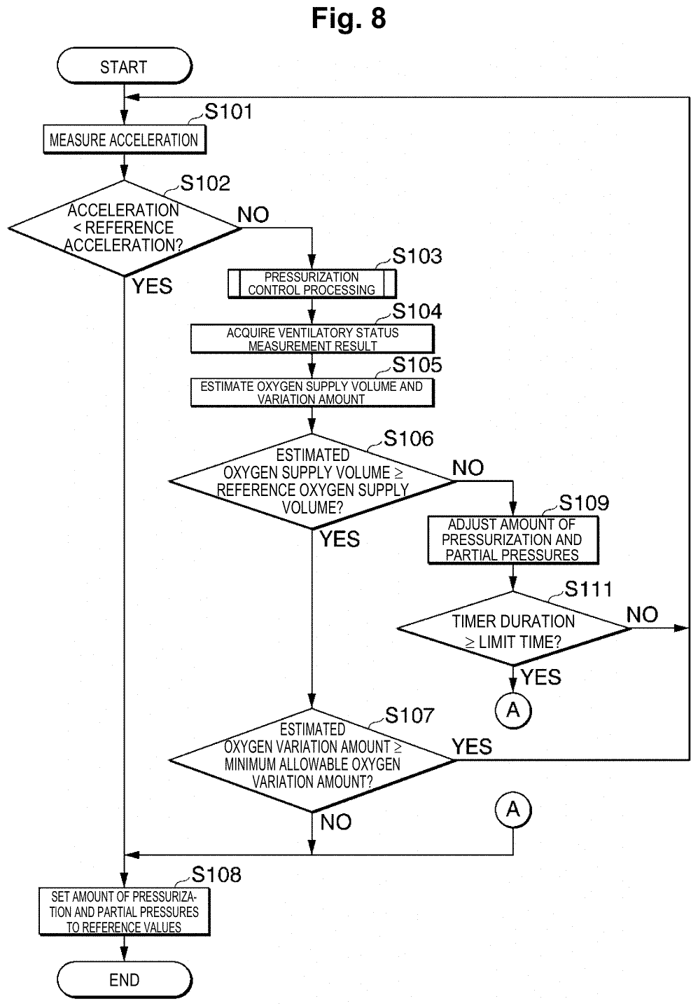

FIG. 8 is a flowchart showing a flow of specific processing where the consciousness disorder mitigation apparatus 1 in the first embodiment includes the second safety mechanism and pressurizes the neck of the user. The flowchart of FIG. 8 is different from the flowchart of FIG. 4 in that there is processing in step S111 after the processing in step S109 in addition to the flowchart of FIG. 4. In the processing of FIG. 8, description of similar processing to FIG. 4 is omitted by giving the same reference numerals. The processing shown in FIG. 8 will be referred to as third consciousness disorder mitigation processing below.

In the following description, a time elapsed since when the pressurization control unit 143 controls the pressurization unit 15 to apply a pressure to the user 91 in step 103 is referred to as timer duration. The timer duration is measured by the timer 150. After step S109, the pressurization control unit 143 and the gas control unit 144 acquire the timer duration and determine whether the timer duration is equal to or greater than the limit time or not (step S111).

If the timer duration is equal to or greater than the limit time (step S111: YES), the processing in step S108 is executed. If the timer duration is a period of time shorter than the limit time (step S111: NO), the flow returns to the processing in step S101.

The consciousness disorder mitigation apparatus 1 with the second safety mechanism in the variant configured as described above can apply a pressure to the user 91 as appropriate for the condition of the user 91 by including the pressurization unit 15 which makes contact with the neck of the user, the impedance measurement unit 142 which measures the impedance of the head of the user 91, and the pressurization control unit 143 which estimates the body fluid volume in the skull of the user 91 and its variation amount per unit time based on the measured impedance and controls the pressurization unit 15 based on the estimated values.

The consciousness disorder mitigation apparatus 1 may also measure the time course and interval of control on the control valve 16 by the control valve control unit 402 via the timer 150 and monitor the gas supply volume from the mask 18 to the lungs per unit time via the gas sensor 182, thereby containing the supply volume within a range not beyond a predefined upper limit volume or lower limit volume of the supply volume of the oxygen gas in accordance with the estimated brain oxygen supply volume mentioned above. With such control, the consciousness disorder mitigation apparatus 1 can also prevent airway disorder due to exposure to high concentration oxygen, for example.

### Second Embodiment

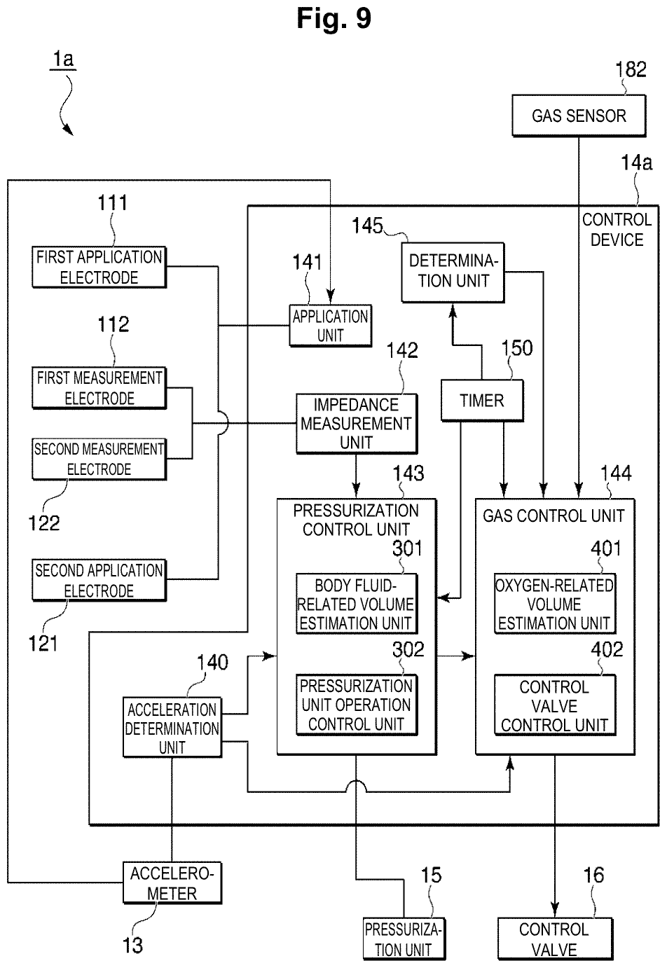

FIG. 9 shows an example of functional configuration of a consciousness disorder mitigation apparatus 1a in a second embodiment. The consciousness disorder mitigation apparatus 1a is different from the consciousness disorder mitigation apparatus 1 in that the consciousness disorder mitigation apparatus 1a includes a control device 14a in place of the control device 14.

The control device 14a is different from the control device 14 including the timer 150 in that the control device 14a includes a determination unit 145. In the following, description of components with similar functions to those of the consciousness disorder mitigation apparatus 1 is omitted by giving the same reference numerals as in FIGS. 1 to 3 and 7.

The determination unit 145 whether a condition that a state of the oxygen supply volume being lower than a predetermined volume continues for a predetermined period of time or longer (hereinafter a “consciousness loss time condition”) is met or not based on the oxygen supply volume and the time measured by the timer 150.

If the determination unit 145 determines that the consciousness loss time condition is met, the control device 14 controls the operation of the oxygen supply unit 100. The control device 14 supplies oxygen gas at a predetermined pressure to the user 91 from the first cylinder 17-1 regardless of the determination result of the acceleration determination unit 140. With supply of oxygen gas at the predetermined pressure to the user 91, the oxygen supply volume is forced to increase.

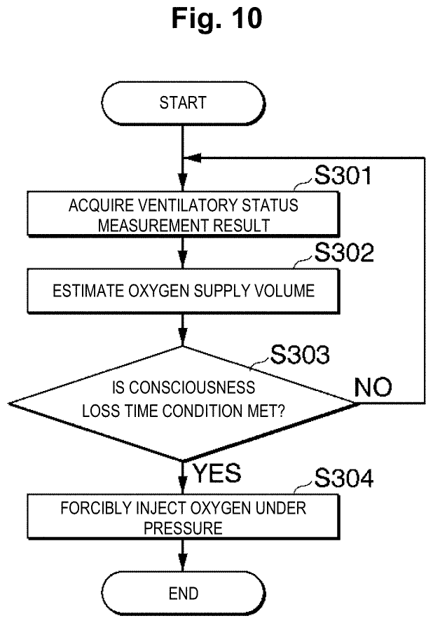

The consciousness disorder mitigation apparatus 1a executes at least one processing of the first consciousness disorder mitigation processing, the second consciousness disorder mitigation processing, and the third consciousness disorder mitigation processing. In parallel, the consciousness disorder mitigation apparatus 1a executes fourth consciousness disorder mitigation processing, shown in FIG. 10. The fourth consciousness disorder mitigation processing is executed during the execution of the first consciousness disorder mitigation processing, the second consciousness disorder mitigation processing and the third consciousness disorder mitigation processing.

FIG. 10 is a flowchart showing an example of flow of processing executed by the consciousness disorder mitigation apparatus 1a in the second embodiment.

The oxygen-related volume estimation unit 401 acquires a ventilatory status measurement result, which is the measurement result of the gas sensor 182 (step S301). After step S301, the oxygen-related volume estimation unit 401 acquires the estimated oxygen supply volume based on the ventilatory status measurement result (step S302).

The determination unit 145 determines whether the consciousness loss time condition is met or not (step S303).

If the consciousness loss time condition is met (step S303: YES), the control valve control unit 402 drives the control valve 16 to inject oxygen gas under pressure to the user 91 from the first cylinder 17-1 (step S304). In step S304, the first consciousness disorder mitigation processing, the second consciousness disorder mitigation processing or the third consciousness disorder mitigation processing is stopped.

If the consciousness loss time condition is not met (step S303: NO), the flow returns to the processing in step S301.

The consciousness disorder mitigation apparatus 1a in the second embodiment configured as described above forcibly increases the oxygen supply volume by injecting oxygen gas under pressure to the user 91 from the first cylinder 17-1 when the duration for which a state of decreased oxygen supply volume continues exceeds a predetermined period of time. When the oxygen supply volume has lowered, the possibility of loss of consciousness occurring increases with time. Also, an abrupt decrease in the oxygen supply volume to brain tissue associated with reduction of oxygen saturation in blood which is caused by disorder of brain blood flow or ventilation disorder gives rise to a consciousness disorder within several seconds and further causes an irreversible disorder, such as brain infarction. Accordingly, the consciousness disorder mitigation apparatus 1a of the second embodiment configured as described above can suppress the occurrence of decline in consciousness or loss of consciousness of the user 91 in association with reduction in the oxygen supply volume in the brain of the user 91.

(Variants)

The consciousness disorder mitigation apparatus 1a may continuously implement a cycle of pressurized injection and pressure reduction (release) of oxygen gas and forcibly repeat the ventilation of the lungs (artificial respiration) by controlling the operation of the control valve 16. By thus repeating the ventilation of the lungs, the consciousness disorder mitigation apparatus 1a in the second embodiment can accelerate the recovery of consciousness of the user 91.

The pressurization unit 15 need not necessarily pressurize the neck only. The pressurization unit 15 may pressurize or decompress a body part other than the neck in coordination with pressurization of the neck. The pressurization unit 15 may pressurize or decompress limbs or the abdomen simultaneously with or at a time difference from the pressurization of the neck, for example.

The consciousness disorder mitigation apparatus 1 and 1a need not necessarily bring the second electrode portion 12 into contact with the neck but may make it contact the abdomen or the waist.

The first electrode portion 11 of the consciousness disorder mitigation apparatus 1 and 1a need not necessarily have two electrodes but may have a single electrode with similar functions as the first application electrode 111 and the first measurement electrode 112. Likewise, the second electrode portion 12 of the consciousness disorder mitigation apparatus 1 and 1a need not necessarily have two electrodes but may have a single electrode with similar functions as the second application electrode 121 and the second measurement electrode 122.

Further, the first electrode portion 11 of the consciousness disorder mitigation apparatus 1 and 1a need not necessarily have two electrodes but may have three or more electrodes.

Furthermore, the second electrode portion 12 of the consciousness disorder mitigation apparatus 1 and 1a need not necessarily have two electrodes but may have three or more electrodes.

While the method of measuring impedance in the embodiments is a so-called 4-terminal method, the impedance would be measured in a so-called 2-terminal method if both the first electrode portion 11 and the second electrode portion 12 have only one electrode.

The consciousness disorder mitigation apparatus 1 and 1a need not necessarily include two electrode portions but may include one or three or more electrode portions.

The application unit 141, the impedance measurement unit 142, the pressurization control unit 143, and the gas control unit 144 need not necessarily implemented in one casing; some or all of the functional units may be separately implemented.

The application of voltage in step S201 of FIG. 5 need not necessarily take place in step S201 but voltage may be applied at any point before the measurement of impedance in step S204.

Between the first measurement electrode 112 and the second measurement electrode 122, a voltage need not necessarily be applied but a current may be applied instead. Also, the impedance measurement unit 142 need not necessarily measure the impedance by acquiring currents flowing in the first measurement electrode 112 and the second measurement electrode 122; the impedance measurement unit 142 may measure the impedance by acquiring voltages flowing in the first measurement electrode 112 and the second measurement electrode 122.

Estimation of the body fluid volume by the consciousness disorder mitigation apparatus 1 and 1a need not necessarily be done by an impedance-based method but may be done by any method that can estimate the body fluid volume in the head of the user 91. For example, the body fluid volume may be estimated from a change in reflectance or transmittance of an electromagnetic wave, such as infrared, applied to the user 91.

For an estimation method with an electromagnetic wave such as infrared, the consciousness disorder mitigation apparatus 1 and 1a may include a light receiving element in place of the first electrode portion 11, the second electrode portion 12 and the impedance measurement unit 142. For an estimation method with an electromagnetic wave such as infrared, the consciousness disorder mitigation apparatus 1 and 1a may include a light source for emitting an electromagnetic wave such as infrared in place of the application unit 141. Further, for an estimation method with an electromagnetic wave such as infrared, the consciousness disorder mitigation apparatus 1 and 1a may include a pressurization control unit 143a in place of the pressurization control unit 143. The light receiving element receives an electromagnetic wave which was emitted by the light source and reflected in the head of the user 91. The light receiving element outputs a signal indicating an intensity of the received light. The pressurization control unit 143a controls the pressure applied by the pressurization unit 15 to the user 91 based on the acceleration signal outputted by the accelerometer 13 and the signal outputted by the light receiving element.

For example, the pressurization control unit 143a estimates the body fluid volume in the skull from the intensity of the electromagnetic wave received by the light receiving element. The body fluid volume estimated by the pressurization control unit 143a is estimated by an estimation method based on the following physical phenomenon: the intensity of electromagnetic wave received by the light receiving element is weaker as more body fluid is present in the skull because the electromagnetic wave such as infrared emitted by the light source is absorbed by the body fluid in the skull.

Further, the method of estimation of the body fluid volume by the consciousness disorder mitigation apparatus 1 and 1a may also be a method of estimation based on ultrasound, aside from electromagnetic wave. In a method of estimation based on ultrasound, diameters of blood vessels in the neck or the head, blood flow, pressure, size of a venous sinus or a cerebrospinal fluid cavity and the like may be measured via ultrasound and the body fluid volume in the head may be estimated based on the resulting measurement data. More specifically, an ultrasound-based method of estimating the body fluid volume may be a method represented by ultrasonic echo, or a method that estimates the body fluid volume from temporal change in reflection points in the body, depth and/or distance, or the body fluid volume may be estimated from flow rate or pressure difference via Doppler echo.

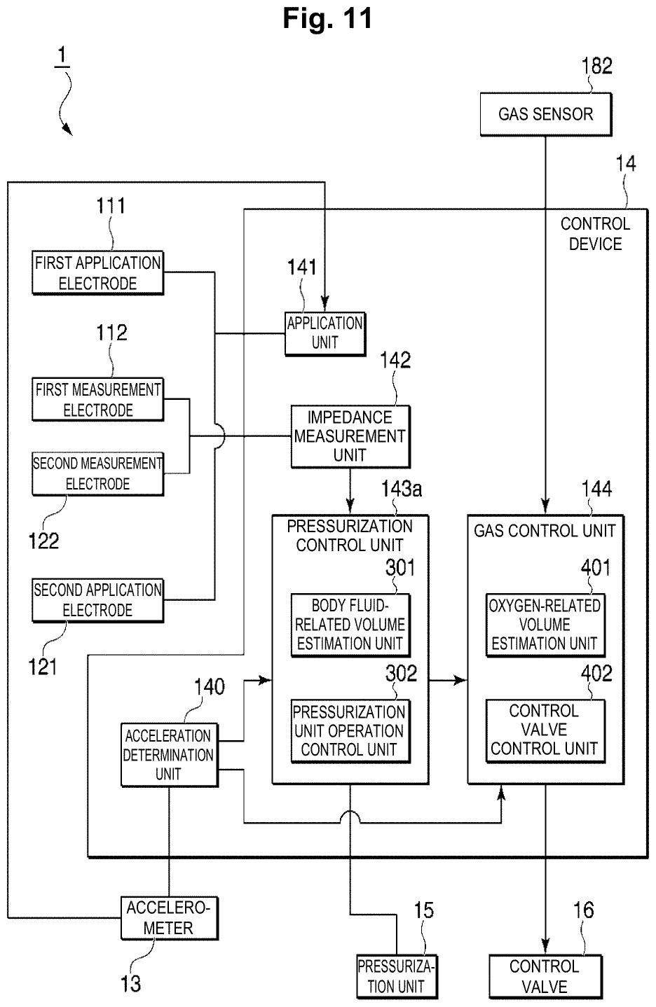

FIG. 11 shows an example of functional configuration of the consciousness disorder mitigation apparatus 1 with the pressurization control unit 143a in a variant.

The consciousness disorder mitigation apparatus 1 shown in FIG. 11 is different from the consciousness disorder mitigation apparatus 1 shown in FIG. 3 in that it includes the pressurization control unit 143a in place of the pressurization control unit 143.

The pressurization control unit 143 and 143a need not necessarily estimate the body fluid volume in the skull and its variation amount per unit time but may estimate only either one of them. Also, the pressurization control unit 143 and 143a need not necessarily control the pressurization unit 15 based on the body fluid volume in the skull and its variation amount per unit time but may control the pressurization unit 15 based on only either one of them.

The oxygen supply unit 100 need not necessarily include one first cylinder 17-1 and one control valve 16 connected to the mask 18 but may include multiple first cylinders 17-1 and control valves 16 connected to the mask 18. In such a case, the control valve 16 that is controlled for its operation in the first consciousness disorder mitigation processing, the second consciousness disorder mitigation processing or the third consciousness disorder mitigation processing and the control valve 16 that is controlled for its operation in the fourth consciousness disorder mitigation processing may be different control valves 16.

(Applications)

The consciousness disorder mitigation apparatus 1 and 1a may be used for the purpose of avoiding dizziness upon standing or a fainting fit associated with reduction in head blood volume. The consciousness disorder mitigation apparatus 1 and 1a may also be used on a subject with lowered anti-G capability who can have a fainting fit in response to a relatively minor change in G such as standing up motion or ascent of an elevator, which is observed with patients who have blood pressure adjustment dysfunction such as Shy-Drager syndrome and diabetic neuropathy. The anti-G capability is ability to stand burden of acceleration on the body, where a user 91 with higher anti-G capability can stand burden of acceleration on the body more. Besides, the consciousness disorder mitigation apparatus 1 and 1a may also be used on operators such as pilots who are in a special high-G environment such as an airplane or drivers, for example.

The body fluid-related volume estimation unit 301 is an example of a first estimation unit. The oxygen-related volume estimation unit 401 is an example of a second estimation unit. The control valve control unit 402 is an example of a partial pressure control unit.

Some or all of the application unit 141, the impedance measurement unit 142, the pressurization control unit 143 and 143a, the gas control unit 144, and the determination unit 145 in the above-described embodiments may be embodied in a computer. In that case, the functions may be embodied by recording a program for embodying the functions on a computer-readable recording medium and loading and executing the program recorded on the computer-readable recording medium into a computer system. A “computer system” as called herein is intended to include an OS and hardware such as peripherals. A “computer-readable recording medium” refers to a storage device such as a removable medium such as a flexible disk, magneto-optical disk, ROM and CD-ROM, or a hard disk incorporated in a computer system. Moreover, a “computer-readable recording medium” may also include a medium that dynamically holds a program for a short time, like a communication line when the program is transmitted over a network such as the Internet or a communication line such as a telephone line, or a medium that holds a program for a certain period of time, like volatile memory within a computer system serving as a server or a client in such a case. Also, the program may be for embodying some of the above-described functions, or further may be capable of embodying the above-described functions in combination with a program already recorded in a computer system, and may be embodied using a programmable logic device such as a FPGA (Field Programmable Gate Array).

While the embodiments of the present invention have been described with reference to the drawings, specific configuration is not limited to those embodiments and designs or the like falling within the scope of the present invention are also encompassed.

### REFERENCE SIGNS LIST

1 consciousness disorder mitigation apparatus
11 first electrode portion
12 second electrode portion
13 accelerometer
14 control device
15 pressurization unit
100 oxygen supply unit
111 first application electrode
112 first measurement electrode
121 second application electrode
122 second measurement electrode
140 acceleration determination unit
141 application unit
142 impedance measurement unit
143, 143a pressurization control unit
144 gas control unit
145 determination unit
151 neck pressurization unit
152 front neck bladder
91 user
92 helmet
93 ear muff
94 internal jugular vein
95 airway

1. A consciousness disorder mitigation apparatus comprising:
a first estimation unit configured to estimate body fluid volume information, the body fluid volume information being information on a body fluid volume present in a head of a user;
a second estimation unit configured to estimate oxygen supply volume information, the oxygen supply volume information being information on an oxygen supply volume representing an amount of oxygen in a brain of the user;
a pressurization unit configured to be attached to the user and to apply a pressure corresponding to an estimation result of the first estimation unit and an estimation result of the second estimation unit; and
an oxygen supply unit configured to supply oxygen to the user based on the estimation result of the second estimation unit.

2. The consciousness disorder mitigation apparatus according to claim 1, further comprising:
a partial pressure control unit configured to adjust a partial pressure of oxygen gas and a partial pressure of non-oxygen gas in mixture gas based on the oxygen supply volume information as the estimation result of the second estimation unit, the mixture gas being mixed gas of the oxygen gas which is oxygen in gas form and the non-oxygen gas which is gas of a different composition from the oxygen,
wherein the oxygen supply unit supplies the mixture gas adjusted by control of the partial pressure control unit to the user.

3. The consciousness disorder mitigation apparatus according to claim 1, wherein the second estimation unit estimates the oxygen supply volume information based on oxygen concentration in expiration or inspiration of the user.

4. The consciousness disorder mitigation apparatus according to claim 1, wherein
the second estimation unit estimates the oxygen supply volume as the oxygen supply volume information, and
when a state in which the oxygen supply volume estimated by the second estimation unit is lower than a predetermined volume continues for a predetermined period of time or longer, the oxygen supply unit supplies oxygen in gas form at a predetermined pressure to the user.

5. A consciousness disorder mitigation method comprising:
a first estimation step of estimating body fluid volume information, the body fluid volume information being information on a body fluid volume present in a head of a user;
a second estimation step of estimating oxygen supply volume information, the oxygen supply volume information being information on an oxygen supply volume representing an amount of oxygen in a brain of the user;
a pressurization step of applying a pressure corresponding to an estimation result from the first estimation step and an estimation result from the second estimation step to the user; and
an oxygen supply step of supplying oxygen to the user based on the estimation result from the second estimation step.
